(19) United States
(12) Patent Application Publication
(10) Pub. No.: US 2022/0292895 A1
(43) Pub. Date: Sep. 15, 2022

(54) Method For Monitoring And Alarming Hydrogen Leakage Of Fuel-Cell Vehicle And System Thereof

(71) Applicants: Tianjin University, Tianjin (CN); China Automotive Technology and Research Center Co., Ltd., Tianjin (CN)

(72) Inventors: Meilin Ren, Tianjin (CN); Tianyou Wang, Tianjin (CN); Fang Wang, Tianjin (CN); Yuntang He, Tianjin (CN); Dong Hao, Tianjin (CN); Zhizhao Che, Tianjin (CN); Zhen Lu, Tianjin (CN); Cheng Wang, Tianjin (CN); Chenshu Hu, Tianjin (CN); Hao Lan, Tianjin (CN)

(21) Appl. No.: 17/739,370

(22) Filed: May 9, 2022

(30) Foreign Application Priority Data
Mar. 10, 2021 (CN) 202110260698.0

(51) Int. Cl. G07C 5/08; G08B 21/18; B60K 15/03; H01M 8/04664; H01M 8/0444; H01M 8/04992; G01N 33/00

(52) U.S. Cl. CPC G07C 5/0816; G08B 21/182; B60K 15/03; H01M 8/04664; H01M 8/04447; H01M 8/04992; G01N 33/0063; B60K 2015/03315; B60K 2015/0321; H01M 2250/20

(57) ABSTRACT

The present disclosure relates to a method for monitoring and alarming hydrogen leakage of a fuel-cell vehicle and a system thereof. The method comprises the steps of: acquiring the current hydrogen concentration of a fuel-cell vehicle; judging whether the current hydrogen concentration is 0, if not, acquiring the measured hydrogen concentration, the hydrogen pressure drop value and the normal pressure range generated when the current hydrogen concentration of the fuel-cell vehicle is 0; judging whether the hydrogen pressure drop value exceeds the normal pressure range, if so, determining the predicted hydrogen concentration according to the measured hydrogen concentration and the hydrogen pressure drop value; judging whether the predicted hydrogen concentration exceeds the hydrogen leakage alarm threshold, and if not, determining the hydrogen leakage and issuing an alarm. The hydrogen concentration alarm limit can be reached in advance, thereby shortening the time for monitoring and alarming hydrogen leakage.

### CROSS REFERENCE TO RELATED APPLICATION

This patent application claims the benefit and priority of Chinese Patent Application No. 202110260698.0, filed on Mar. 10, 2021, the disclosure of which is incorporated by reference herein in its entirety as part of the present application.

### TECHNICAL FIELD

The present disclosure relates to the field of monitoring and alarming hydrogen leakage of a fuel-cell vehicle, in particular to a method for monitoring and alarming hydrogen leakage a fuel-cell vehicle and a system thereof.

### BACKGROUND ART

Hydrogen leakage detection of a fuel cell system can directly determine whether there is hydrogen leakage by measuring the pressure drop of hydrogen pressure or the concentration of hydrogen leakage. However, the existing hydrogen leakage detection method can only obtain the leakage situation unilaterally through the hydrogen concentration or pressure change, which has many restrictive factors, slow reaction speed and low monitoring efficiency. The measurement reaction time of the hydrogen concentration sensor is long, and the fluctuation of hydrogen pressure is random and easy to give false alarms in the actual process of fuel cell operation.

### SUMMARY

The purpose of the present disclosure is to provide a method for monitoring and alarming hydrogen leakage of a fuel-cell vehicle and a system thereof, so as to solve the problem in the prior art that the hydrogen leakage cannot be quickly monitored due to the long response time of a sensor after the hydrogen leakage of the fuel-cell vehicle.

To achieve the above purpose, the present disclosure provides the following scheme.

The present disclosure relates to a method for monitoring and alarming hydrogen leakage of a fuel-cell vehicle, wherein the method for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the method for monitoring and alarming hydrogen leakage comprises the steps of:

acquiring the current hydrogen concentration of a fuel-cell vehicle;

judging whether the current hydrogen concentration is 0 to obtain a first judgment result;

if the first judgment result indicates that the current hydrogen concentration is not 0, acquiring the measured hydrogen concentration, the hydrogen pressure drop value and the normal pressure range generated when the current hydrogen concentration of the fuel-cell vehicle is 0;

judging whether the hydrogen pressure drop value exceeds the normal pressure range to obtain a second judgment result;

if the second judgment result indicates that the hydrogen pressure drop value exceeds the normal pressure range, determining the predicted hydrogen concentration according to the measured hydrogen concentration and the hydrogen pressure drop value;

judging whether the predicted hydrogen concentration exceeds the hydrogen leakage alarm threshold to obtain a third judgment result;

if the third judgment result indicates that the predicted hydrogen concentration exceeds the hydrogen leakage alarm threshold, determining the hydrogen leakage and issuing an alarm.

Preferably, determining the predicted hydrogen concentration according to the measured hydrogen concentration and the hydrogen pressure drop value specifically comprises:

determining the predicted hydrogen concentration according to the formula C_{REV}(t)=a (t)*ΔP (t)*C_{H2}(t); in which C_{REV} is the predicted hydrogen concentration; a(t) is the concentration correction coefficient; ΔP(t) is the hydrogen pressure drop value; C_{H2} is the measured hydrogen concentration.

Preferably, if the first judgment result indicates that the current hydrogen concentration is 0, a normal initial pressure drop value is acquired;

a normal pressure range is generated according to the normal initial pressure drop value.

Preferably, if the second judgment result indicates that the hydrogen pressure drop value does not exceed the normal pressure range, the measured hydrogen concentration and the hydrogen pressure drop value are re-acquired.

Preferably, if the third judgment result indicates that the predicted hydrogen concentration does not exceed the hydrogen leakage alarm threshold, the measured hydrogen concentration and the hydrogen pressure drop value are re-acquired.

The present disclosure relates to a system for monitoring and alarming hydrogen leakage of a fuel-cell vehicle, wherein the system for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the system for monitoring and alarming hydrogen leakage comprises:

a current hydrogen concentration acquiring module, which is configured to acquire the current hydrogen concentration of a fuel-cell vehicle;

a first judging module, which is configured to judge whether the current hydrogen concentration is 0 to obtain a first judgment result;

a parameter acquiring module, which is configured to, if the first judgment result indicates that the current hydrogen concentration is not 0, acquire the measured hydrogen concentration, the hydrogen pressure drop value and the normal pressure range generated when the current hydrogen concentration of the fuel-cell vehicle is 0;

a second judging module, which is configured to judge whether the hydrogen pressure drop value exceeds the normal pressure range to obtain a second judgment result;

a predicted hydrogen concentration determining module, which is configured to, if the second judgment result indicates that the hydrogen pressure drop value exceeds the normal pressure range, determine the predicted hydrogen concentration according to the measured hydrogen concentration and the hydrogen pressure drop value;

a third judging module, which is configured to judge whether the predicted hydrogen concentration exceeds the hydrogen leakage alarm threshold to obtain a third judgment result;

a hydrogen leakage determining module, which is configured to, if the third judgment result indicates that the predicted hydrogen concentration exceeds the hydrogen leakage alarm threshold, determine the hydrogen leakage and issue an alarm.

Preferably, the predicted hydrogen concentration determining module specifically comprises:

a predicted hydrogen concentration determining unit, which is configured to determine the predicted hydrogen concentration according to the formula C_{REV}(t)=a (t)*ΔP (t)*C_{H2} (t); in which C_{REV} is the predicted hydrogen concentration; a(t) is the concentration correction coefficient; ΔP(t) is the hydrogen pressure drop value; C_{H2} is the measured hydrogen concentration.

Preferably, the system further comprises:

a normal initial pressure drop value acquiring module, which is configured to, if the first judgment result indicates that the current hydrogen concentration is 0, acquire a normal initial pressure drop value;

a normal pressure range generating module, which is configured to generate a normal pressure range according to the normal initial pressure drop value.

Preferably, the system further comprises:

a first parameter reacquiring module, which is configured to, if the second judgment result indicates that the hydrogen pressure drop value does not exceed the normal pressure range, re-acquire the measured hydrogen concentration and the hydrogen pressure drop value.

Preferably, the system further comprises:

a second parameter reacquiring module, which is configured to, if the third judgment result indicates that the predicted hydrogen concentration does not exceed the hydrogen leakage alarm threshold, re-acquire the measured hydrogen concentration and the hydrogen pressure drop value.

According to the specific embodiment according to the present disclosure, the present disclosure discloses the following technical effects. The present disclosure provides a method for monitoring and alarming hydrogen leakage of a fuel-cell vehicle and a system thereof. When hydrogen leaks, a hydrogen pressure sensor measures pressure fluctuation, and a high-sensitivity hydrogen concentration sensor measures tiny hydrogen leakage at the same time, and determines the predicted hydrogen concentration weighted by a pressure drop algorithm according to the amplitude of pressure fluctuation (i.e., hydrogen pressure drop value), so that the hydrogen concentration alarm limit can be reached in advance, thereby shortening the time for monitoring and alarming hydrogen leakage.

### BRIEF DESCRIPTION OF THE DRAWINGS

In order to explain the embodiments of the present disclosure or the technical scheme in the prior art more clearly, the drawings needed in the embodiments will be briefly introduced hereinafter. Obviously, the drawings in the following description are only some embodiments of the present disclosure. For those skilled in the art, other drawings can be obtained according to these drawings without paying creative labor.

FIG. 1 is a schematic diagram of the distribution of hydrogen concentration sensors and hydrogen pressure sensors in a fuel-cell vehicle;

FIG. 2 is a simplified flow chart of monitoring and alarming hydrogen leakage of a fuel-cell vehicle;

FIG. 3 is a flow chart of a method for monitoring and alarming hydrogen leakage of a fuel-cell vehicle according to the present disclosure;

FIG. 4 is a flow chart of another method for monitoring and alarming hydrogen leakage of a fuel-cell vehicle according to the present disclosure;

FIG. 5 is a structural diagram of a system for monitoring and alarming hydrogen leakage of a fuel-cell vehicle according to the present disclosure.

### DETAILED DESCRIPTION OF THE EMBODIMENTS

The technical scheme in the embodiments of the present disclosure will be described clearly and completely hereinafter with reference to the drawings in the embodiments of the present disclosure. Obviously, the described embodiments are only some embodiments of the present disclosure, rather than all of the embodiments. Based on the embodiments of the present disclosure, all other embodiments obtained by those skilled in the art without paying creative labor belong to the scope of protection of the present disclosure.

The purpose of the present disclosure is to provide a method for monitoring and alarming hydrogen leakage of a fuel-cell vehicle and a system thereof, in which the hydrogen concentration alarm limit can be reached in advance, thereby shortening the time for monitoring and alarming hydrogen leakage.

In order to make the above objects, features and advantages of the present disclosure more obvious and understandable, the present disclosure will be further explained in detail hereinafter with reference to the drawings and specific embodiments.

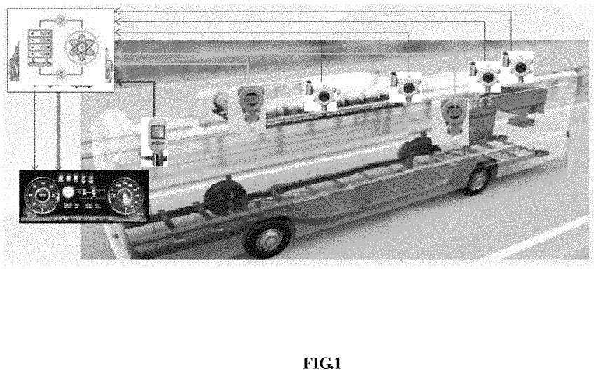

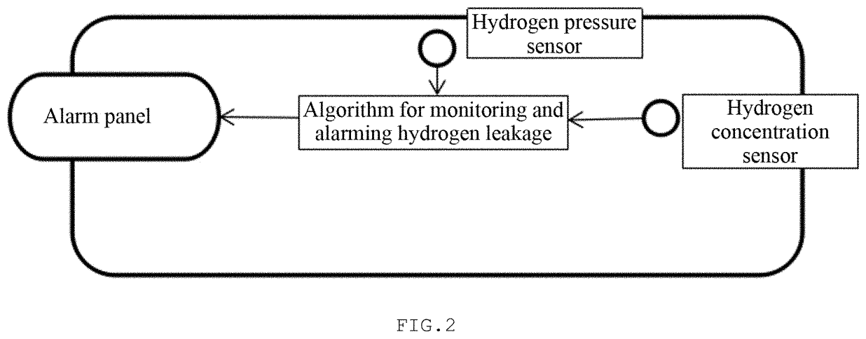

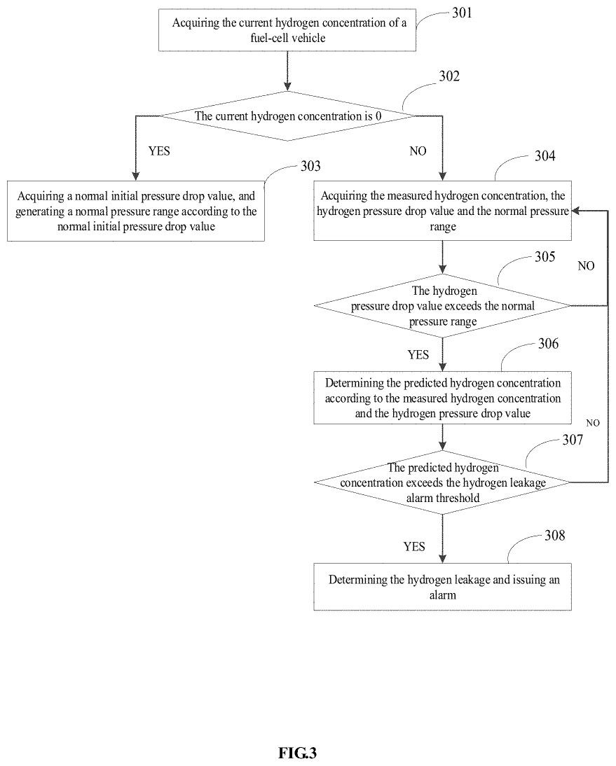

FIG. 1 is a schematic diagram of the distribution of hydrogen concentration sensors and hydrogen pressure sensors in a fuel-cell vehicle. FIG. 2 is a simplified flow chart of monitoring and alarming hydrogen leakage of a fuel-cell vehicle. FIG. 3 is a flow chart of a method for monitoring and alarming hydrogen leakage of a fuel-cell vehicle according to the present disclosure. As shown in FIGS. 1-3, a method for monitoring and alarming hydrogen leakage of a fuel-cell vehicle is provided, wherein the method for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the method for monitoring and alarming hydrogen leakage comprises the steps of:

Step 301: acquiring the current hydrogen concentration of a fuel-cell vehicle;

Step 302: judging whether the current hydrogen concentration is 0, if so, proceeding to step 303, otherwise, proceeding to step 304;

Step 303: acquiring a normal initial pressure drop value, and generating a normal pressure range according to the normal initial pressure drop value;

Step 304: acquiring the measured hydrogen concentration, the hydrogen pressure drop value and the normal pressure range generated when the current hydrogen concentration of the fuel-cell vehicle is 0;

Step 305: judging whether the hydrogen pressure drop value exceeds the normal pressure range, if so, proceeding to step 306, otherwise, returning to step 304;

Step 306: determining the predicted hydrogen concentration according to the measured hydrogen concentration and the hydrogen pressure drop value;

Step 306 specifically comprises: determining the predicted hydrogen concentration according to the formula C_{REV}(t)=a (t)*ΔP (t)*C_{H2}(t); in which C_{REV} is the predicted hydrogen concentration; a(t) is the concentration correction coefficient; ΔP(t) is the hydrogen pressure drop value; C_{H2} is the measured hydrogen concentration. The algorithm for monitoring and alarming hydrogen leakage in FIG. 2 is C_{REV}(t)=a (t)*ΔP (t)*C_{H2}(t).

Step 307: judging whether the predicted hydrogen concentration exceeds the hydrogen leakage alarm threshold, if so, proceeding to step 308, and if not, returning to step 304.

Step 308: determining the hydrogen leakage and issuing an alarm.

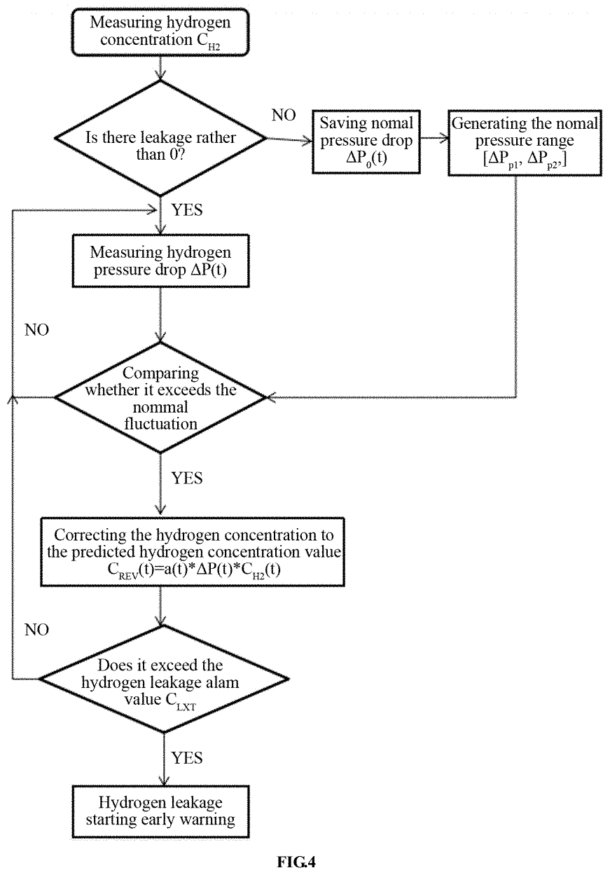

FIG. 4 is a flow chart of another method for monitoring and alarming hydrogen leakage of a fuel-cell vehicle according to the present disclosure, as shown in FIG. 4, which specifically comprises the following steps:

Step 1: ensuring that the vehicle is in normal condition and the hydrogen system has no leakage, i.e. C_{H2}=0, and under the actual vehicle working condition:

Step 2: collecting the pressure fluctuation value ΔP(t) of the hydrogen pressure sensor;

Step 3: generating the normal hydrogen pressure fluctuation range [ΔP₁, ΔP₂] of the system and storing it in the whole vehicle controller;

Step 4: after the vehicle is powered on, detecting the hydrogen pressure fluctuation ΔP(t) and the hydrogen concentration C_{H2} by the whole vehicle in real time;

Step 5: if it is detected that the hydrogen pressure fluctuation ΔP(t) exceeds [ΔP₁, ΔP₂], correcting the measured hydrogen concentration value to C_{REV}: C_{REV}(t)=a (t)*ΔP (t)*C_{H2}(t).

Step 6: judging whether the corrected hydrogen concentration value is greater than the alarm limit C_{LMT}, if so, starting an alarm, otherwise, continuing monitoring.

When the vehicle operates normally, the vehicle monitors the hydrogen pressure drop value of the vehicle, and forms different normal pressure drop fluctuation ranges [ΔP₁, ΔP₂] under different operating conditions. When the detected pressure fluctuation exceeds [ΔP₁, ΔP₂], the hydrogen concentration sensor for hydrogen leakage is triggered to monitor ultra-quickly, that is to say, C_{REV} value is used instead of C_{H2} value, so as to achieve early and rapid monitoring and alarming of hydrogen leakage.

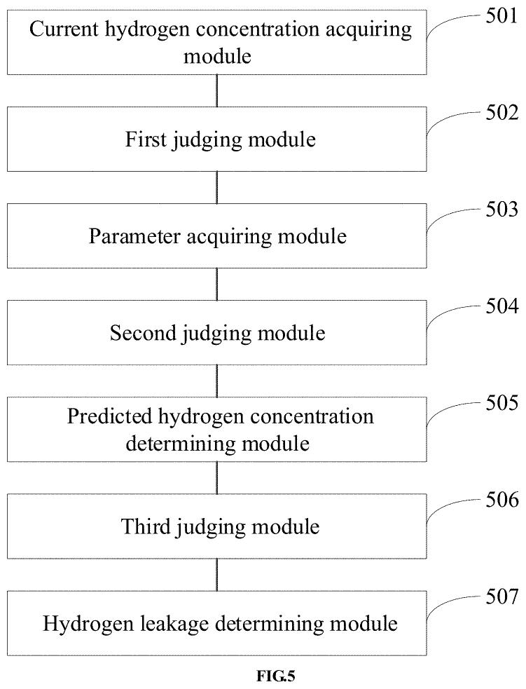

FIG. 5 is a structural diagram of a system for monitoring and alarming hydrogen leakage of a fuel-cell vehicle according to the present disclosure. As shown in FIG. 5, a system for monitoring and alarming hydrogen leakage of a fuel-cell vehicle is provided, wherein the system for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the system for monitoring and alarming hydrogen leakage comprises:

a current hydrogen concentration acquiring module 501, which is configured to acquire the current hydrogen concentration of a fuel-cell vehicle;

a first judging module 502, which is configured to judge whether the current hydrogen concentration is 0 to obtain a first judgment result;

a parameter acquiring module 503, which is configured to, if the first judgment result indicates that the current hydrogen concentration is not 0, acquire the measured hydrogen concentration, the hydrogen pressure drop value and the normal pressure range generated when the current hydrogen concentration of the fuel-cell vehicle is 0;

a second judging module 504, which is configured to judge whether the hydrogen pressure drop value exceeds the normal pressure range to obtain a second judgment result;

a predicted hydrogen concentration determining module 505, which is configured to, if the second judgment result indicates that the hydrogen pressure drop value exceeds the normal pressure range, determine the predicted hydrogen concentration according to the measured hydrogen concentration and the hydrogen pressure drop value;

the predicted hydrogen concentration determining module 505 specifically comprises: a predicted hydrogen concentration determining unit, which is configured to determine the predicted hydrogen concentration according to the formula C_{REV}(t)=a (t)*ΔP (t)*C_{H2} (t); in which C_{REV} is the predicted hydrogen concentration; a(t) is the concentration correction coefficient; ΔP(t) is the hydrogen pressure drop value; C_{H2} is the measured hydrogen concentration;

a third judging module 506, which is configured to judge whether the predicted hydrogen concentration exceeds the hydrogen leakage alarm threshold to obtain a third judgment result;

a hydrogen leakage determining module 507, which is configured to, if the third judgment result indicates that the predicted hydrogen concentration exceeds the hydrogen leakage alarm threshold, determine the hydrogen leakage and issue an alarm.

The present disclosure further comprises: a normal initial pressure drop value acquiring module, which is configured to, if the first judgment result indicates that the current hydrogen concentration is 0, acquire a normal initial pressure drop value; a normal pressure range generating module, which is configured to generate a normal pressure range according to the normal initial pressure drop value.

The present disclosure further comprises: a first parameter reacquiring module, which is configured to, if the second judgment result indicates that the hydrogen pressure drop value does not exceed the normal pressure range, re-acquire the measured hydrogen concentration and the hydrogen pressure drop value.

The present disclosure further comprises: a second parameter reacquiring module, which is configured to, if the third judgment result indicates that the predicted hydrogen concentration does not exceed the hydrogen leakage alarm threshold, re-acquire the measured hydrogen concentration and the hydrogen pressure drop value.

The method and system for monitoring and alarming according to the present disclosure can shorten the time for monitoring and alarming hydrogen leakage of s fuel-cell vehicle, and improve the alarming efficiency of a fuel cell, thereby enhancing the safety of the hydrogen fuel-cell vehicle. The present disclosure can be installed and implemented on fuel-cell buses on a large scale, so as to greatly improve the safety of hydrogen use of vehicles in operation.

In this specification, each embodiment is described in a progressive manner, and each embodiment focuses on the differences from other embodiments. It is sufficient to refer to the same and similar parts among each embodiment. Because the system disclosed in the embodiment corresponds to the method disclosed in the embodiment, the description is relatively simple, and the relevant points can be found in the description of the method.

In the present disclosure, a specific example is applied to illustrate the principle and implementation of the present disclosure, and the explanation of the above embodiments is only used to help understand the method and its core idea of the present disclosure. At the same time, according to the idea of the present disclosure, there will be some changes in the specific implementation and application scope for those skilled in the art. To sum up, the contents of this specification should not be construed as limiting the present disclosure.

What is claimed is:

1. A method for monitoring and alarming hydrogen leakage of a fuel-cell vehicle, wherein the method for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the method for monitoring and alarming hydrogen leakage comprises the steps of:
acquiring the current hydrogen concentration of a fuel-cell vehicle;
judging whether the current hydrogen concentration is 0 to obtain a first judgment result;
if the first judgment result indicates that the current hydrogen concentration is not 0, acquiring the measured hydrogen concentration, the hydrogen pressure drop value and the normal pressure range generated when the current hydrogen concentration of the fuel-cell vehicle is 0;
judging whether the hydrogen pressure drop value exceeds the normal pressure range to obtain a second judgment result;
if the second judgment result indicates that the hydrogen pressure drop value exceeds the normal pressure range, determining the predicted hydrogen concentration according to the measured hydrogen concentration and the hydrogen pressure drop value;
judging whether the predicted hydrogen concentration exceeds the hydrogen leakage alarm threshold to obtain a third judgment result;
if the third judgment result indicates that the predicted hydrogen concentration exceeds the hydrogen leakage alarm threshold, determining the hydrogen leakage and issuing an alarm.

2. The method for monitoring and alarming hydrogen leakage of a fuel-cell vehicle according to claim 1, wherein determining the predicted hydrogen concentration according to the measured hydrogen concentration and the hydrogen pressure drop value specifically comprises:
determining the predicted hydrogen concentration according to the formula C_{REV}(t)=a (t)*ΔP (t)*C_{H2} (t); in which C_{REV} is the predicted hydrogen concentration; a(t) is the concentration correction coefficient; ΔP(t) is the hydrogen pressure drop value; C_{H2} is the measured hydrogen concentration.

3. The method for monitoring and alarming hydrogen leakage of a fuel-cell vehicle according to claim 1, wherein if the first judgment result indicates that the current hydrogen concentration is 0, a normal initial pressure drop value is acquired;
a normal pressure range is generated according to the normal initial pressure drop value.

4. The method for monitoring and alarming hydrogen leakage of a fuel-cell vehicle according to claim 1, wherein if the second judgment result indicates that the hydrogen pressure drop value does not exceed the normal pressure range, the measured hydrogen concentration and the hydrogen pressure drop value are re-acquired.

5. The method for monitoring and alarming hydrogen leakage of a fuel-cell vehicle according to claim 1, wherein if the third judgment result indicates that the predicted hydrogen concentration does not exceed the hydrogen leakage alarm threshold, the measured hydrogen concentration and the hydrogen pressure drop value are re-acquired.

6. A system for monitoring and alarming hydrogen leakage of a fuel-cell vehicle, wherein the system for monitoring and alarming hydrogen leakage is applied to a fuel-cell vehicle, hydrogen concentration sensors are arranged at the top positions of a hydrogen storage area, an engine compartment area and a passenger compartment area of a fuel cell of a fuel-cell vehicle; hydrogen pressure sensors are installed on the mouth valve of a hydrogen bottle and a main pressure reducing valve of a hydrogen system; the system for monitoring and alarming hydrogen leakage comprises:
a current hydrogen concentration acquiring module, which is configured to acquire the current hydrogen concentration of a fuel-cell vehicle;
a first judging module, which is configured to judge whether the current hydrogen concentration is 0 to obtain a first judgment result;
a parameter acquiring module, which is configured to, if the first judgment result indicates that the current hydrogen concentration is not 0, acquire the measured hydrogen concentration, the hydrogen pressure drop value and the normal pressure range generated when the current hydrogen concentration of the fuel-cell vehicle is 0;
a second judging module, which is configured to judge whether the hydrogen pressure drop value exceeds the normal pressure range to obtain a second judgment result;
a predicted hydrogen concentration determining module, which is configured to, if the second judgment result indicates that the hydrogen pressure drop value exceeds the normal pressure range, determine the predicted hydrogen concentration according to the measured hydrogen concentration and the hydrogen pressure drop value;
a third judging module, which is configured to judge whether the predicted hydrogen concentration exceeds the hydrogen leakage alarm threshold to obtain a third judgment result;
a hydrogen leakage determining module, which is configured to, if the third judgment result indicates that the predicted hydrogen concentration exceeds the hydrogen leakage alarm threshold, determine the hydrogen leakage and issue an alarm.

7. The system for monitoring and alarming hydrogen leakage of a fuel-cell vehicle according to claim 6, wherein the predicted hydrogen concentration determining module specifically comprises:
a predicted hydrogen concentration determining unit, which is configured to determine the predicted hydrogen concentration according to the formula C_{REV}(t)=a (t)*ΔP (t)*C_{H2}(t) in which C_{REV} is the predicted hydrogen concentration; a(t) is the concentration correction coefficient; ΔP(t) is the hydrogen pressure drop value; C_{H2} is the measured hydrogen concentration.

8. The system for monitoring and alarming hydrogen leakage of a fuel-cell vehicle according to claim 6, further comprising:
a normal initial pressure drop value acquiring module, which is configured to, if the first judgment result indicates that the current hydrogen concentration is 0, acquire a normal initial pressure drop value;
a normal pressure range generating module, which is configured to generate a normal pressure range according to the normal initial pressure drop value.

9. The system for monitoring and alarming hydrogen leakage of a fuel-cell vehicle according to claim 6, further comprising:
a first parameter reacquiring module, which is configured to, if the second judgment result indicates that the hydrogen pressure drop value does not exceed the normal pressure range, re-acquire the measured hydrogen concentration and the hydrogen pressure drop value.

10. The system for monitoring and alarming hydrogen leakage of a fuel-cell vehicle according to claim 6, further comprising:
a second parameter reacquiring module, which is configured to, if the third judgment result indicates that the predicted hydrogen concentration does not exceed the hydrogen leakage alarm threshold, re-acquire the measured hydrogen concentration and the hydrogen pressure drop value.
